(19) United States
(12) Patent Application Publication
(10) Pub. No.: US 2010/0172981 A1
(43) Pub. Date: Jul. 8, 2010

(54) Ibuprofen-Effervescent Preparation Having a High Dissolution Rate and Method for the Production thereof

(75) Inventors: Peter Gruber, Merzhausen (DE); Hubert Keck, Freiburg (DE)

Correspondence Address: HOXIE & ASSOCIATES LLC, 75 MAIN STREET , SUITE 301, MILLBURN, NJ, 07041, US

(73) Assignee: Losan Pharma GMBH, Nuenburg (DE)

(21) Appl. No.: 12/443,107

(22) PCT Filed: Aug. 30, 2007
(86) PCT No.: PCT/EP07/59043

§ 371 Date: Jan. 11, 2010

(30) Foreign Application Priority Data
Sep. 26, 2006 (EP) 06121278.3

(51) Int. Cl. A61K 9/46; A61K 9/14; A61K 31/192; A61P 29/00

(52) U.S. Cl. 424/466; 424/489; 514/570

(57) ABSTRACT

Effervescent ibuprofen formulations, which contain (a) an active ingredient-containing granule which contains a water-soluble ibuprofen salt and a basic excipient, and (b) an effervescent granule which contains an acid component and a carbon dioxide-forming component, and a process for their preparation.

The invention relates to effervescent formulations with the active ingredient ibuprofen which dissolve quickly in water accompanied by the formation of a clear solution, and to a simplified process for their preparation.

Ibuprofen (2-(4-isobutylphenyl) propionic acid) is a known non-steroidal active ingredient that has proved successful over many years, with an analgesic and anti-inflammatory action. It is used, inter alia, for the treatment of headaches and pain, swellings and fevers caused by inflammation.

A particularly rapid build-up of blood level in ibuprofen is desired when treating pain. It has been shown with ibuprofen that an increased plasma ibuprofen level leads to an increased analgesic effect. Examination of numerous dosage forms showed that dissolved ibuprofen leads to an earlier start to pain relief than do dragées or film-coated tablets.

It is known that, of all dosage forms, effervescent tablets, dissolved in water, lead most rapidly to the build-up of a high blood level of the active ingredient. Therefore they are indicated particularly if rapid and safe pain relief is desired. The dissolved active ingredient is transported quickly by the relatively large volume of liquid via the stomach into the upper intestinal tract, where the ibuprofen is substantially resorbed.

However, ibuprofen is an organic acid and virtually insoluble in water. Only in the pH range from approx. 6 does it gradually dissolve through salt formation. A rapid and safe dissolution of the ibuprofen is particularly important when developing an effervescent tablet, because undissolved particles of active ingredient continue to adhere to the mucous membranes upon drinking. As a consequence of the neutral to weakly alkaline pH of saliva these particles dissolve slowly, wherein in addition to an unpleasant taste burning and local irritations on the mucous membranes occur.

The preparation of soluble salts of ibuprofen and their use in effervescent tablets or granules which are dissolved in water is the subject of numerous patents and patent applications.

DE 36 38 414 A1 discloses effervescent compositions with the active ingredient ibuprofen which contain the strongly basic amino acids arginine or lysine in a quantity exceeding the molar portion to improve the dissolution of the ibuprofen. It is pointed out that the arginine and lysine salts of ibuprofen are not suitable for the preparation of effervescent compositions, as they do not lead to a complete dissolution of the ibuprofen. The compositions contain sodium hydrogen tartrate as acid constituent. Arginine and lysine clearly cost more than the active ingredient ibuprofen and are therefore too expensive for use as pharmaceutical excipients.

EP 0 369 228 A1 discloses an effervescent ibuprofen formulation which contains basic granular material containing an active ingredient, and an acid component. To prepare the basic granular material a water-soluble ibuprofen salt is granulated together with a carrier and a stabilizer, the granule sprayed with a sodium or potassium carbonate solution and then dried. Preferred ibuprofen salts are the potassium and in particular the sodium salt. The preparation of the basic granule is laborious and expensive. The embodiment example shows that the preparation of ibuprofen granules for 100,000 tablets requires a total of 230 kg water which is evaporated again in a fluid-bed granulator at 100° C. A further problem is that the water content of effervescent tablets must be less than 0.5 wt.-% if the tablets are stored in packs with a desiccant, and less than 0.25 wt.-% when using blister packs.

The preparation of water-soluble alkali-metal salts of ibuprofen is known for example from U.S. Pat. No. 4,859,704. For this, ibuprofen is suspended in water and neutralized for example by adding sodium or potassium hydrogen carbonate. During this reaction, the respective ibuprofen salt is formed and carbon dioxide and water released. The release of carbon dioxide is a major problem during industrial-scale production. For example, if 400 kg ibuprofen is reacted in the described manner, 44,800 litres of carbon dioxide form. This makes it necessary to conduct the reaction cautiously and slowly.

A further disadvantage is the necessary drying of the product. The sodium salt of ibuprofen forms a dihydrate which contains 13.6 wt.-% water of crystallization and is not suitable for the preparation of a stable effervescent tablet, as the water of crystallization is not bound firmly enough. Over time it is partly given off and brings about a reaction of the effervescing body in the effervescent tablet. Thus, either the effervescent tablets must be stored in packs with a desiccant, or the water of crystallization removed before processing, which is extremely costly and time-consuming. Moreover, ibuprofen-sodium tends to adhere to the tabletting moulds and can therefore be pressed into tablets only with difficulty, which makes its processing difficult and makes the use of large quantities of excipients necessary. A further disadvantage is that ibuprofen-sodium dissolves only slowly in water. Additionally, ibuprofen-sodium is approximately three times more expensive than the free acid. The potassium salt of ibuprofen is extremely hygroscopic, with the result that its drying likewise involves a considerable outlay. Because of its hygroscopicity this salt is not available on the world market in commercial quantities.

WO 94/10994 discloses effervescent ibuprofen tablets based on water-soluble ibuprofen salts such as e.g. the sodium salt which are characterized in that the active ingredient has a crystal size of 180 to 800 μm. The relatively large crystal size is supposed to simplify the preparation of the tablets. Crystals with the desired size are obtained by dissolving ibuprofen in a large excess of denatured alcohol followed by neutralization with sodium hydroxide. The use of hydroxides leads to the formation of water, which in addition to the large quantity of alcohol must be removed by drying. A large quantity of effervescing body is necessary to dissolve the only slowly-dissolving sodium salt of the ibuprofen. To prepare the tablets, 1600 mg sodium hydrogen carbonate is therefore used per 200 mg ibuprofen, and the tablet weight is approximately 2300 mg in total. The tablets are thus relatively large and therefore expensive to prepare. Additionally, the sodium salt resulting from the reaction of the sodium hydrogen carbonate with the acid and the pH connected thereto of the effervescent tablet solution produces a salty, unpleasant, soapy taste. EP 0 667 149 A1 describes effervescent ibuprofen tablets which contain sodium carbonate, sodium hydrogen carbonate and potassium hydrogen carbonate. To prepare the tablets the active ingredient or a salt thereof is granulated with the basic components and then mixed with an acid granule which is prepared separately therefrom. The mixture is then formed into tablets. The granulation takes place with water, which necessitates an expensive drying. The tablets are supposed to dissolve in water within two minutes.

U.S. Pat. No. 6,171,617 discloses effervescent ibuprofen formulations which contain two separate granules, (a) ibuprofen-containing granules and (b) effervescent granules which contain an acid component and a carbon dioxide-forming component. To prepare the active ingredient granule, ibuprofen is mixed with a basic excipient and then granulated with water or a water-alcohol mixture. Sodium carbonate, potassium carbonate, sodium hydrogen carbonate, potassium hydrogen carbonate and trisodium phosphate are named as preferred basic excipients. The water is removed again by drying after the preparation of the granule. The two granules are mixed and packed in pouches or pressed into tablets. The tablets weigh 3.3 g with an active ingredient quantity of 200 mg. The active ingredient-containing granule (a) contains ibuprofen in the form of its acid and not as a water-soluble salt. A staggered dissolution of the granules is thereby to be achieved. Firstly, the effervescent granule (b) dissolves and only then the active ingredient itself.

Furthermore effervescent tablets are known which contain ibuprofen lysinate or ibuprofen arginate as active ingredients. These salts are expensive and little suited to the preparation of effervescent tablets, as they do not lead to a complete dissolution of the ibuprofen (DE 36 38 414)).

The above statements show that the preparation of effervescent ibuprofen formulations is time-consuming and expensive. Therefore, although the active ingredient ibuprofen possesses clear advantages vis-à-vis comparable active ingredients such as acetylsalicylic acid and paracetamol, there are no inexpensive pleasant-tasting effervescent tablets on the market. An effervescent tablet with 400 mg ibuprofen was briefly available but was not accepted because of its unpleasant taste, while a 600 mg effervescent tablet has thus far not even been offered for sale.

The object of the invention is to make available effervescent ibuprofen formulations which dissolve quickly in water accompanied by the formation of a clear solution. The solution is further to have a pleasant taste and able to be economically produced. A further object of the invention is the provision of a process which makes possible the rapid and cheap preparation of such tablets. In particular, the process is to require no expensive drying steps.

This object is achieved according to the invention by effervescent ibuprofen formulations which
(a) contain an active ingredient-containing granule which contains ibuprofen and a basic excipient, and
(b) contain an effervescent granule which contains an acid component and a carbon dioxide-forming component.

The formulations are characterized in that the active ingredient-containing granule contains the ibuprofen in the form of a water-soluble salt, preferably the potassium salt of ibuprofen or a mixture of the potassium and sodium salt. Formulations which contain the active ingredient ibuprofen exclusively in the form of the potassium salt or of a mixture of the potassium and the sodium salts are particularly preferred. The potassium salt and the sodium salt are preferably used in a molar ratio of potassium to sodium salt of 1:0 to 1:1, particularly preferably 1:0.02 to 1:0.1, quite particularly preferably 1:0.05.

Ibuprofen exists in two stereoisomeric forms, the R(−) form and the S(+) form. The R(−) form is substantially less pharmacologically active than the S(+) form. Unless otherwise stated, by ibuprofen or ibuprofen salt is meant the respective racemate. All given quantities relate to the racemate, wherein in the case of salts, unless otherwise stated, the corresponding quantity of free acid is listed. In addition to the racemate, the use of the S(+) form is preferred. This is used in half the quantity of the racemate. Unless otherwise stated, by ibuprofen is meant herein the free ibuprofen acid.

As basic component the active ingredient-containing granule preferably contains a base (base 2) which forms a first base (base 1) during the reaction with ibuprofen. During this reaction, ibuprofen changes into the corresponding ibuprofen salt, and base 1 forms base 2 by absorbing a proton.

In the case of the basic substance described below preferred as base 1, there forms as base 2 NaHCO₃, KHCO₃, dipotassium citrate, disodium citrate, disodium phosphate, dipotassium phosphate, the protonated forms of lysine, arginine, physiologically acceptable, basic organic amines, such as meglumine (N-methylglucamine).

As base 1 is preferably used in excess, the basic component can also contain mixtures of base 1 and base 2 and optionally also mixtures of different bases 1 and/or bases 2. The active ingredient-containing granule preferably contains as basic component KHCO₃, a mixture of KHCO₃ and K₂CO₃, a mixture of KHCO₃ and NaHCO₃ or a mixture of KHCO₃, K₂CO₃, NaHCO₃ and Na₂CO₃.

The ratio of ibuprofen salt to basic excipient in the active ingredient-containing granule lies preferably in the range from 0.8 to 2 mol, particularly preferably 1 to 1.5 mol and quite particularly preferably 1.1 to 1.25 mol basic excipient per mol ibuprofen salt.

In addition to the water-soluble ibuprofen salt and the basic excipient the active ingredient-containing granule can contain further excipients, for example substances to improve solubility, wettability, pressibility and flow behaviour. Preferred further excipients are binders, such as povidone and cellulose ester, neutral water-soluble excipients, such as sorbitol, mannitol, maltitol, isomaltitol, weakly basic excipients. Overall, however, the aim is to use no, or only small quantities of, further excipients in addition to the above-named basic excipients. Granules which contain no further excipients in addition to the ibuprofen salt and the basic excipients are therefore quite particularly preferred.

The effervescent formulation contains as second component an effervescent granule which preferably contains as acid component a physiologically harmless, inorganic or preferably organic edible acid or an acid salt thereof, in particular citric acid, tartaric acid, malic acid, ascorbic acid, an acid salt of these acids, such as monosodium citrate, monosodium tartrate or monosodium fumarate, an acid inorganic salt, such as monopotassium phosphate, potassium hydrogen sulphate or sodium hydrogen sulphate, glutamic acid, adipic acid, glutamic acid hydrochloride, betaine hydrochloride or a mixture thereof.

Preferred carbon dioxide-forming components are alkaline or alkaline-earth carbonates or hydrogen carbonates, in particular NaHCO₃, NaCO₃, KHCO₃, K₂CO₃, sodium glycine carbonate and mixtures thereof, quite particularly preferably NaHCO₃, KHCO₃ or a mixture thereof.

In addition to the acid component and the carbon dioxide-eliminating component the effervescent granule usually contains further excipients, for example binders such as povidone or methylhydroxypropyl celluloses, in order to guarantee well-structured effervescing bodies that do not form dust. The effervescing bodies can also contain neutral excipients which dissolve well in water, such as sucrose, glucose, sorbitol, mannitol, xylitol and maltitol, which serve to control the reactivity of the effervescing body.

For better wetting of the constituents of the formulation and to accelerate decomposition, the effervescent formulations according to the invention preferably contain at least one physiologically acceptable surfactant. Surfactants with a HLB value >12, preferably 12 to 18, particularly preferably 14 to 16, such as e.g. sodium lauryl sulphate or sucrose palmitate are particularly suitable. The surfactant(s) is (are) preferably used in a quantity of 0.01 to 0.1 parts by weight, preferably 0.025 to 0.035 parts by weight per part by weight ibuprofen. The surfactants can be contained both in the active-ingredient granule and also in the effervescent granules, but preferably are added to effervescent granules.

Furthermore the formulations for improving the taste can contain sweeteners and flavourings which are water-soluble or dispersible in water. In addition to sugars such as sucrose, sweeteners are preferred in particular as they have a high sweetening strength and are thus particularly suitable for the preparation of small, cheaper effervescent tablets. Preferred sweeteners are aspartame, sodium sacchrinate, sodium cyclamate, acesulfame K, neohesperidin, sucralose and mixtures of the named substances. Sweeteners are preferably used in a quantity of 5 to 100 mg, preferably 10 to 50 mg and quite preferably 20 to 30 mg per 200 mg ibuprofen.

Preferred flavourings are peppermint, menthol and vanilla flavourings, particularly citrus flavourings such as e.g. grapefruit flavouring. Flavourings are preferably used in a quantity of less than 100 mg, preferably less than 75 mg per 200 mg ibuprofen. Flavourings are preferably used in a quantity of 10 to 100 mg, preferably 15 to 75 mg and quite preferably 25 to 40 mg per 200 mg ibuprofen.

The active ingredient-containing granules and/or the effervescent granule can, in addition to ibuprofen, also contain further active ingredients, for example antihistamines, anti-mucositic active ingredient, antacids, analgesics such as aspirin or paracetamol, expectorants, anaesthetic active ingredients and combinations thereof. Particularly preferred active ingredients are diphenhydramine, chlorpheniramine maleate, brompheniramine maleate, phenylpropanolamine, phenylephrine hydrochloride, pseudoephedrine hydrochloride, codeine, ascorbic acid and caffeine.

The effervescent formulations are preferably available in the form of a dosing unit, particularly preferably in the form of effervescent tablets or drinkable granules packed in sachets or stickpacks, i.e. an effervescent ibuprofen granule which contains the active-ingredient granule (a) and effervescent granule (b) e.g. in the form of a mixture.

The active ingredient-containing granule (a) is characterized by a high solubility and in particular dissolution rate, with the result that the active ingredient-containing granule and the effervescent granule dissolve at practically the same time. The dissolution time is clearly reduced compared with granules with staggered dissolution. The dissolution rate of the active ingredient-containing granules is preferably 5 to 20 seconds, particularly preferably 5 to seconds and quite particularly preferably 5 to 12 seconds, with the result that the granule scarcely reaches the bottom of the measurement container. The particle size of the granule is preferably in the range from 0.1 to 1.25 mm, particularly preferably 0.1 to 0.9 mm.

Because of these preferred properties the quantity of effervescing body necessary for the preparation of an effervescent tablet dissolving in 2 to 3 minutes can be substantially reduced. For example effervescent tablets can be prepared which weigh less than 1.5 g in a dose of 200 mg ibuprofen, but nevertheless clearly dissolve in water and have a pleasant taste because of the small quantity of effervescing body and in particular sodium hydrogen carbonate. Thus effervescent tablets are also accessible which have an acceptable taste even with an active ingredient quantity of 600 mg ibuprofen.

The dissolution rate of granules is measured with a dissolution apparatus according to the European Pharmacopoeia. For this the so-called paddle method is used in which 500 ml demineralized water at a temperature of 20° C. is poured into a vessel. The contents of the vessel are stirred at a speed of 100 revolutions per minute, and 1.0 g of the granule to be measured is added.

Effervescent tablets must, according to their monograph in all the pharmacopoeias, dissolve practically on their own without stirring. Thus the effervescent component also serves to ensure that any formed sediment is dissolved by a sufficiently strong CO₂ formation.

The formulation according to the invention preferably contains 200 to 800 mg ibuprofen (measured as free acid) per dosing unit. Effervescent tablets preferably contain 200, 400 or 600 mg ibuprofen, sachets or stickpacks 200, 400, 600 or 800 mg ibuprofen per dosing unit.

In the case of effervescent tablets the dosing units preferably have a total weight of 1000 to 1500 mg per 200 mg ibuprofen (measured as free acid). Preferred total weights for a dosing unit are accordingly:
200 mg ibuprofen: 1000 to 1500 mg, preferably 1300 to 1400 mg;
400 mg ibuprofen: 2000 to 3000 mg, preferably 2400 to 2800 mg;
600 mg ibuprofen: 3000 to 4500 mg, preferably 3500 to 3900 mg.

The formulations according to the invention also preferably contain a total of 3 to 5 parts by weight, particularly preferably 3.5 to 4.2 parts by weight of basic components such as alkali hydrogen carbonate and in particular sodium hydrogen carbonate per part by weight ibuprofen.

The formulations according to the invention also preferably contain 1 to 2 parts by weight, particularly preferably 1.4 to 1.8 parts by weight acid components such as citric acid and/or monosodium citrate per part by weight ibuprofen.

In the case of effervescent tablets, effervescent granule (b) and active-ingredient granule (a) are preferably used in a ratio of 1.7 to 2.9 parts by weight effervescent granule (b), particularly preferably 2.1 to 2.6 parts by weight effervescent granule (b) per 1 part by weight active-ingredient granule (a).

The formulations contain the indicated components in quantities such that the ready-to-drink solution obtained by dissolution of a dosing unit in 200 ml water has a pH of 6.3 to 7.5, preferably <7, in particular 6.5 to 6.8 and quite particularly preferably 6.6 to 6.7, with the result that the solution tingles on the tongue because of the carbon dioxide physically dissolved in the water and has neither a salty nor a soapy taste.

The formulations according to the invention thus differ from known effervescent formulations not only by virtue of their particularly low weight per dosing unit but also by a substantially better, non-salty or -soapy taste. Moreover, the formulations produce completely clear solutions after dissolution.

In the case of drinkable granules which are packed in sachets or stickpacks, the quantity of effervescent granule (b) can be further reduced vis-à-vis the active-ingredient granule (a) with the result that the named advantages are particularly pronounced. Drinkable granules preferably have a total weight of 500 to 950 mg per 200 mg ibuprofen (measured as free acid) per dosing unit. Preferred total weights for a dosing unit of drinkable ibuprofen granule are accordingly:
200 mg ibuprofen: 500 to 950 mg, preferably 550 to 800 mg;
400 mg ibuprofen: 1000 to 1900 mg, preferably 1100 to 1600 mg;
600 mg ibuprofen: 1500 to 2850 mg, preferably 1700 to 2400 mg;
800 mg ibuprofen: 2000 to 3800 mg, preferably 2300 to 3200 mg.

In the case of drinkable granules, the quantity ratio of active-ingredient granules (a) to effervescent granules (b) preferably lies in the range from 0.4 to 1.6 parts by weight effervescent granule (b), preferably 0.6 to 1.25 parts by weight effervescent granule (b) per part by weight active-ingredient granule (a).

The object forming the basis of the invention is also to make available an economical process for the preparation of effervescent ibuprofen formulations. An essential core of the achievement of this object is a process which makes possible the rapid and economical conversion of the insoluble ibuprofen into a highly water-soluble granule even under production conditions.

According to the invention the active ingredient-containing granule (a) is preferably prepared by intensively mixing ibuprofen in the form of its free acid with one or more basic excipients (base 1) without adding water.

The ibuprofen is preferably mixed with the basic excipient while adding an organic solvent. Particularly suitable organic solvents are isopropanol, ethanol, acetone and mixtures thereof, quite particularly isopropanol. The organic solvent is preferably used in a quantity of 0 to 10 wt.-%, particularly preferably 0.5 to 3 wt.-% and quite particularly preferably 0.8 to 1.5 wt.-% relative to the total weight of the reaction mixture. To accelerate the reaction the mixture can be heated, preferably to a temperature in the range of 50 to 60° C.

It was wholly surprisingly found that ibuprofen reacts completely when intensively mixed with the basic excipients without the addition of water. After adding a very small quantity of an organic solvent the mixture heats through its own reaction heat to approx. 40 to 55° C., changing completely into a highly water-soluble ibuprofen-salt mixture.

If for example 360 kg ibuprofen, 270 kg potassium carbonate, 9 kg sodium carbonate and 6 kg isopropanol (0.9%) are mixed at room temperature in a suitable mixing vessel, the product temperature rises to 40 to 50° C. within approximately 30 minutes. The product is initially powdery throughout the process and then changes into a very fine granule without the mixture becoming sticky or losing its flowability. Surprisingly, the reaction proceeds in solid state. If a sample is taken from the stirred mixture after approximately 40 minutes, this dissolves quantitatively in water within a few seconds and produces a completely clear solution.

If a vacuum granulator is used for this process, the isopropanol used can be very largely removed within under 5 minutes as a result of the product heat and the vacuum. Thus the whole process for converting 360 kg ibuprofen (sufficient for 1.8 million effervescent tablets with 200 mg ibuprofen) is over in less than 45 minutes. Even 600 kg ibuprofen can be reacted and dried in less than 60 minutes. The speed of the reaction and in particular the clarity of the course of reaction were unforeseeable and are completely unexpected.

Water-soluble basic substances which have a pH>11.0 in 0.1 molar aqueous solution are suitable as basic excipients (base 1). Preferred basic excipients (base 1) are tripotassium phosphate, trisodium phosphate, tripotassium citrate, trisodium citrate and the corresponding sodium salts, the potassium and sodium salts of glycine, lysine, arginine and physiologically harmless organic amines such as meglumine (N-methylglucamine), in particular alkali carbonates such as sodium carbonate and potassium carbonate and mixtures of these substances.

The basic excipients are preferably used in a quantity of 0.8 to 2 mol, preferably 1.0 to 1.5 mol and quite preferably 1.1 to 1.25 mol basic excipient per mol ibuprofen, wherein potassium-containing basic excipients and particularly mixtures of sodium-containing and potassium-containing basic excipients are preferred. Potassium and sodium salts, such as for example K₂CO₃ and Na₂CO₃, are preferably used in a molar ratio (K:Na) of 1:0 to 1:1, preferably 1:0.02 to 1:0.1, particularly preferably 1:0.05.

Particularly preferred basic excipients are potassium carbonate and mixtures of potassium carbonate and sodium carbonate. In addition to these basic excipients further basic excipients can also be added, which are preferably selected from the group of the above-named basic excipients (base 1). Further basic excipients are preferably used in a quantity of 0 to 0.5 mol of the named total quantity of basic excipients of 0.8 to 2.0 mol per mol ibuprofen.

The average particle size of the ibuprofen and of the alkaline excipients should not be greater than 0.18 mm, preferably not greater than 0.125 mm and quite particularly preferably not greater than 0.1 mm. At least 95% of all the components participating in the reaction should be smaller than 0.25 mm. For reasons of cost, in all cases particles with a minimum average particle size of 0.025 mm and particularly 0.05 mm are preferred. The average particle size is ascertained according to DIN 66145, i.e. from the Rosin-Rammler-Sperling-Bennet (RRSB) curve known to a person skilled in the art.

The ibuprofen reacts with the basic compound(s) (base 1) accompanied by the formation of an intimate salt mixture which contains the ibuprofen salt and the reaction product (base 2) of the basic compound 1 and where appropriate excesses of the base 1. When using potassium carbonate as basic compound 1 ibuprofen potassium and potassium hydrogen carbonate forms as base 2. It is completely unexpected that under the described mild reaction conditions the crystals of the ibuprofen and of the basic excipient through-react totally into the molecular state accompanied by the formation of salt without a solution or a melt visibly occurring. An extremely intimate, very homogeneous mixture between the ibuprofen salt and the basic excipient forms, wherein mixtures which contain ibuprofen-potassium and potassium hydrogen carbonate are quite particularly preferred. Both the potassium salt of ibuprofen and the potassium hydrogen carbonate formed are very well soluble in water and contribute to the high solubility and the extremely high dissolution rate of the active ingredient-containing granule according to the invention. The active ingredient-containing granule can also have small voids which presumably result from the inclusion of CO₂. These voids have diameters of for example 10 to 40 μm, in particular approx. 30 μm and can give the granules a honeycomb-like structure.

It was also found that during the preparation or dissolution of the effervescent formulation the hydrogen carbonate intimately connected to the ibuprofen salt almost completely suppresses the reverse reaction to insoluble ibuprofen of the active-ingredient salt with the acid contained in the effervescent granule. This is a decisive cause of the clear dissolution of the effervescent ibuprofen formulations according to the invention and for the surprisingly low pH of the resulting effervescent solution.

According to a preferred embodiment of the process according to the invention a mixture of potassium and sodium salts, in particular a mixture of potassium carbonate and sodium carbonate, is used as basic excipient. Although the ibuprofen-sodium resulting from the addition of sodium carbonate is less well soluble than the potassium salt, a very well soluble ibuprofen-salt mixture is obtained. The addition of a small quantity of a sodium compound has the advantage that the resultant ibuprofen sodium acts as a binder because of its adhesiveness and improves the processibility of the active-ingredient granule.

Moreover, in the absence of water, anhydrous ibuprofen-sodium forms which, because of its tendency to form a dihydrate tends to absorb water and binds water pulled in for example by the starting substances or formed in small quantities accompanied by the formation of ibuprofen-sodium dihydrate. Thus it acts as an internal desiccant. Under the production conditions customary for the preparation of effervescent tablets of approx. 20 to 25° C. and approx. 20% relative humidity the granule is sufficiently stable and absorbs only insignificant quantities of water from the air. The granule prepared in the vacuum granulator can be screened and mixed without problems.

The ibuprofen-salt mixtures prepared according to the invention are characterized in addition to their high dissolution rate by better pressibility and also by a small tendency to stick.

The water content of the granule is to lie if at all possible in the range from <0.5 wt.-%, particularly preferably <0.25 wt.-%. The granule is therefore dried if necessary following the reaction. A water content of <0.5 wt.-% and in particular 0.25 wt.-% is necessary to guarantee a sufficient storage stability of the effervescent formulations. These must be stable over several years and must not show any carbon dioxide development through reaction with the effervescent granule if exposed to short-term temperature stress.

Granules with a water content of <0.5 wt.-% are particularly suitable for effervescent formulations which are packaged in packs with a desiccant, for example in tubes with a drying plug. When using e.g. blister packs or alu/alu foils the water content should lie below 0.25 wt.-%.

The prevention of the addition of water is essential for the course of the reaction. Therefore, basic excipients which form water upon reaction with the ibuprofen acid, such as e.g. KOH and NaOH or also potassium hydrogen carbonate and sodium hydrogen carbonate, are preferably not used. Bicarbonates have the additional disadvantage that during the reaction with an acid, in addition to water, carbon dioxide also forms.

The drying of hydrous granules is energy-intensive and costly. To achieve a water content of less than 0.5 wt.-% more than 20 hours are required under production conditions for a 500-kg mixture even under vacuum when water is used during the preparation of the active ingredient-containing granule. A further problem, recognized only under production conditions on the 500-kg scale, is that in the presence of water the reaction of ibuprofen with carbonates does not proceed uniformly. Although the ibuprofen reacts preferably with the more basic carbonate accompanied by the formation of hydrogen carbonate, in the presence of water a reaction also takes place with the more weakly basic hydrogen carbonate accompanied by the formation of carbon dioxide according to the following reaction diagram:

Ibuprofen+K₂CO₃→ibuprofen-potassium+KHCO₃

KHCO₃+ibuprofen→ibuprofen-potassium+CO₂+H₂O

The extent of these reactions depends on the mixing intensity, the temperature of the product and other parameters and can thus be controlled only with difficulty under production conditions, with the result that non-reproducible losses of mass occur. The composition of every batch must be analyzed and re-adjusted accordingly to the found level of active ingredient. The drying is additionally made difficult by the fact that sodium hydrogen carbonate already starts to decompose from a temperature of approximately 60° C. accompanied by the formation of water and carbon dioxide, whereupon the above problems are further intensified. The water present, which dissolves the carbonates and the hydrogen carbonates used, which then react with the ibuprofen insoluble in water, bears sole responsibility for this behaviour.

According to the invention it was found, on the other hand, that the reaction proceeds reproducibly in the absence of water even on the 600-kg scale and an active ingredient-containing granule with an active ingredient content between 100 and 101% (relative to the theoretical value) results. This small difference is presumably caused by the reaction to form CO₂ and water of small quantities of hydrogen carbonates formed during the process and/or a small concentration of the granule by drying the components used undried. As practically no increase in the concentration of the ibuprofen takes place, it is to be concluded that, to the surprise of a person skilled in the art, practically only carbonate reacts with the ibuprofen and takes up a proton accompanied by the formation of hydrogen carbonate. The reaction, observed when water is added under production conditions, of hydrogen carbonates with ibuprofen does not take place, and fluctuating, non-reproducible active-ingredient concentrations caused thereby do not occur. As the reaction and drying preferably proceed at temperatures below 60° C., the thermal decomposition of formed sodium hydrogen carbonate in CO₂ and water is also excluded.

The process according to the invention can be carried out in any suitable mixing vessel. The mixing is preferably carried out in a vacuum granulator, compactor, fluid-bed reactor or extruder.

When using organic solvents the use of a vacuum granulator is particularly advantageous, in particular a vacuum granulator which, in addition to the devices for mixing the components, also provides for the possibility of immediate drying. The small quantities of solvents which are used according to the invention can be removed in a few minutes in a vacuum granulator. Compactors, fluid-bed reactors or extruders are preferably used for the pure thermal solubilization of the ibuprofen without solvent. The mixture, essentially consisting of ibuprofen and basic excipients, is heated to approx. 50° C. accompanied by stirring. Under these conditions a batch size of approx. 500 kg can be converted within 2 hours into an active-ingredient granule well soluble in water.

In the case of solvent-free conversion in a compactor the mixture of ibuprofen and the named alkaline excipients is strongly compacted between two rolls. The resulting reaction heat is sufficient to carry out the reaction, additional heating is not necessary.

Surprisingly, the thermal reaction of the ibuprofen with the named alkaline excipients can also take place in a fluidized bed. The mixture is heated to 50 to 70° C. by the process air.

According to a further preferred variant the solvent-free reaction can be carried out in an extruder. In this case, ibuprofen and alkaline excipients are accurately dosed into an extruder by means of gravimetrically operating dosing scales and mixed together. The extrusion is preferably carried out under the conditions described below. The segments of the extruder in which the thorough mixing takes place are heated to 60 to 100° C. The adjoining segments of the extruder are cooled with the result that the composition advantageously leaves the extruder as granular, low-dust product at a temperature of approx. 40° C. As a consequence of the higher temperature a limited thermal decomposition of sodium hydrogen carbonate and/or reaction of hydrogen carbonate with ibuprofen can optionally take place here. The extruded material generally has an active-ingredient content of 101.5 to 102.5%. Once the product temperature exceeds approx. 65° C. a small reaction of hydrogen carbonates with still unconverted ibuprofen must be expected, and in particular in the case of sodium hydrogen carbonate a thermal decomposition. Despite the higher reaction temperature this secondary reaction takes place to only a surprisingly small extent. The water content of the extruded material is usually of the order of 0.5 wt.-%.

Granules which have small voids and which have a honeycomb-like structure can be prepared by extrusion. Such granules are particularly suitable for the preparation of drinkable granules.

The active ingredient-containing granule is then mixed with an effervescent granule and then preferably packed in sachets or stickpacks or compressed to form tablets.

The effervescent granule (b) is prepared in a separate step, preferably in a vacuum granulator. An acid component is mixed with a carbon dioxide-forming component and optionally further excipients and additives. The above-named preferred acid components and carbon dioxide-forming components are preferably used. The quantities of acid components and the quantities of CO₂-forming constituents are fixed in a manner known to a person skilled in the art such that the resulting effervescent formulation produces the desired pH when dissolved in water.

A person skilled in the art can control the reactivity of the effervescing body in known manner by selecting well water-soluble and less well water-soluble acid constituents for the effervescent granule. This can e.g. take place in proven manner by mixing a well water-soluble citric acid and relatively poorly soluble monosodium citrate. The dissolution rate of the acid component and thus the reactivity of the effervescent granule can also be controlled by the crystal size e.g. of the citric acid. Naturally, the decomposition time of the effervescent tablets is controlled not only via the reactivity of the effervescing body, but also via the quantity used.

The above-named further excipients are customarily used when preparing the effervescent granules. In order that a reaction between the acid component and the CO₂-forming component does not already occur during granulation, either the constituents of the effervescent granule must be granulated with organic solvents such as e.g. ethanol and isopropanol or only very small quantities of water may be added which are to be removed again as quickly as possible from the dampened effervescent body granules e.g. by applying a vacuum.

As already described, the CO₂-forming components, in particular sodium hydrogen carbonate, are reacted with the acid constituents, preferably essentially citric acid and monosodium citrate, to form an effervescent-body granule. However, acid and basic components can also be added individually to the final mixture. For example, small quantities of an acid and/or a CO₂-forming component can also be added to adjust the pH of the resulting effervescence solution or to accelerate the effervescent reaction of the final mixture. The named quantities of CO₂-forming components and acid constituents are present at the level of 60 to 100 wt.-%, preferably 75 to 90 wt.-% as effervescent granule, the remainder is optionally added to the final mixture.

The invention is explained further below by means of embodiment examples.

### EMBODIMENT EXAMPLES

### Example 1

### Preparation of an Ibuprofen-Containing Granule with K₂CO₃/Na₂CO₃ (1:0.042) Accompanied by the Addition of Isopropanol

360 kg ibuprofen with an average grain of 0.075 mm, 270 kg potassium carbonate with an average grain of 0.09 mm, 9 kg sodium carbonate with an average grain of 0.05 mm were poured into a 1400-litre vacuum granulator and mixed for approx. 5 minutes. 7.5 kg isopropanol was sprayed in within 10 minutes via two 0.5 mm single-component nozzles. The product was continuously stirred over a further 30 minutes, wherein the product temperature increased from 22 to a maximum of 53° C. The product remained constantly powdery during stirring and changed within 40 minutes into a fine, free-flowing granule with an average grain of approx. 0.08 mm. A representative sample of the granule dissolved clear in water within seconds. The conversion of the difficulty soluble ibuprofen into a highly water-soluble ibuprofen-potassium salt mixture was over. A vacuum was then applied for 5 minutes, the product temperature dropped to 35-40° C. and the loss on drying was a 0.5% (essentially isopropanol). This granule can already be used to prepare effervescent tablets. If the drying time was extended to 15 minutes, a loss on drying of less than 0.2% was achieved.

To prepare a coarser ibuprofen granule suitable e.g. for use in 400- or 600-mg effervescent tablets for packing in sachets and stickpacks, the material was granulated by spraying further isopropanol. A sprayed-in quantity of 10-25 kg sufficed. After brief vacuum drying the resulting coarser granule was screened and then dried to the desired loss on drying. The drying time for a loss on drying of approx. 0.5% was approx. 25 minutes, for a loss on drying of <0.2% approx. 60 minutes. The average grain of the granule was 0.25 mm with a grain-size distribution between 0.1 and 1.25 mm.

1.17 mol of the basic components potassium carbonate and sodium carbonate was used per one mol ibuprofen, the ratio of the potassium-containing to the sodium-containing basic excipients being 1:0.042.

The active-ingredient content of the granule was 100.5% (relative to the theoretical set point of 56.3%).

### Example 2

### Determination of the Dissolution Rate of the Granule from Example 1

In a dissolution apparatus, 500 ml demineralized water at 20° C. was introduced first into the vessels according to

European Pharmacopoeia (method 2). The speed of the paddle was set at 100 revolutions per minute. The dissolution time for the granules prepared according to (a) was:

| | |
|---|---|
| average grain 0.08 mm | 8 seconds |
| average grain 0.25 mm | 19 seconds. |

A representative sample of 1 g was used in each case.

### Example 3

### Preparation of an Active Ingredient-Containing Granule with K₂CO₃/Na₂CO₃ (1:0.96) Accompanied by the Addition of Isopropanol

Exactly as per example 1, 412 kg ibuprofen, 155 kg potassium carbonate, 115 kg sodium carbonate, 32 kg mannitol were poured into the vacuum granulator and mixed for 5 minutes. 8 kg isopropanol was sprayed in accompanied by stirring and stirred for 55 minutes. After 50 minutes the composition reached a temperature of 54° C. After approx. 30 minutes' stirring time the stirred mass became slightly sticky for a few minutes and changed into a fine granule after a further 10 minutes. A small quantity of coarser granule agglomerates of the order of up to 2.5 cm formed with the result that after a short drying time of 3 minutes the composition was screened through a sieve with a mesh width of 2.5 mm and then of 1.0 mm. The sieved granule was returned into the vacuum granulator and dried within 45 minutes to a loss on drying of <0.3%.

1.1 mol basic components were used per 1 mol ibuprofen. The ratio of potassium-containing to sodium-containing basic excipients is 1:0.96. The dissolution time, determined according to example 2, of 1 g of the ibuprofen granule is 28 seconds (average grain 0.4 mm). Solvent quantity (relative to total mass) 1.1%.

In example 3 the ibuprofen content was 101.2% (relative to the theoretical set point of 57.7%). This content proves that the reaction proceeded practically exclusively via the carbonates alone. The potassium hydrogen carbonate and sodium hydrogen carbonate formed during the reaction practically did not react with ibuprofen accompanied by the formation of CO₂ and water.

### Example 4

### Preparation of an Active Ingredient-Containing Granule without the Addition of Organic Solvent in a Roller-Compactor

200 kg (970 mol) ibuprofen, 140 kg (1013 mol) potassium carbonate and 24 kg trisodium phosphate (anhydrous, 145.5 mol) were mixed and poured evenly into the funnel of a roller-compactor. Through the action of the pressure (approx. 10 KN) and the heat that formed in the process, including the reaction heat, during the compacting a composition was formed which shortly after cooling could be screened through sieves with mesh widths of 2.5 and 1.25 mm and was completely soluble in water. The two compactor-rollers were water-cooled.

1.19 mol alkaline excipients were used per 1 mol ibuprofen. The ratio of the alkali potassium to sodium salts is 1:0.15. The granule dissolved in 22 seconds in the apparatus described under example 2 under the same test conditions. The average grain was 0.6 mm. The ibuprofen granule is best suited to the preparation of effervescent tablets or for packing into sachets, optionally together with further excipients, and for use as flavourings and sweeteners.

### Example 5

### Preparation of an Active Ingredient-Containing Granule without the Addition of Organic Solvent in the Vacuum Granulator

The following were poured, in succession, into a heatable and coolable 1400-litre vacuum granulator:

412 kg ibuprofen (2000 mol), 276.0 kg potassium carbonate (2000 mol), 14.6 kg lysine (100 mol). The heating jacket of the vacuum granulator was set at 50° C. During a stirring time of 90 minutes the product temperature rose to 68° C. The product was completely water-soluble at this time. A vacuum was applied for 10 minutes and then cooling took place to a product temperature of 35° C. A fine granule with some larger agglomerates formed. Therefore the material was sieved through a sieve with a mesh width of 1.25 mm.

The ratio of ibuprofen to water-soluble alkaline excipients was 1:1.05. The ibuprofen content was 102.7% (relative to 58.7% ibuprofen in the starting mixture). The increase in the content suggests that during the reaction which took place at a somewhat higher temperature small quantities of the formed potassium hydrogen carbonate reacted with ibuprofen accompanied by the formation of CO₂ and water.

A representative 1 g sample dissolved according to the method described in example 2 in 500 ml water at 20° C. within 12 seconds (average particle size 0.2 mm). The granule is best suited to the preparation of effervescent tablets or for the preparation of granules which are dissolved before being taken in water.

No water or solvent was added to the reaction mixture.

### Example 6

### Preparation of an Active Ingredient-Containing Granule with K₂CO₃/Na₂CO₃ (1:0.092) in the Extruder

41.2 kg ibuprofen and a mixture of 30.0 kg potassium carbonate and 2.1 kg sodium carbonate were fed per hour into the 1^{st} segment of a twin-screw extruder. The 2^{nd}-7^{th} segments of the twin-screw extruder were set at a reaction temperature of 100° C. In the 6^{th} segment of the extruder 20 kg maltitol per hour was gravimetrically added. The 8^{th}-10^{th} segments of the extruder were set at 30° C. to cool the solubilized product. The average residence time of the added ibuprofen and the water-soluble basic excipients in the extruder was approx. 2 minutes. At the extruder end a still weakly sticky granule was discharged which after a few minutes could be screened through a sieve with a mesh width of 2.0 mm.

The extruded mass was extremely clearly water-soluble. The ratio of ibuprofen to alkaline excipients was 1:1.19, the ratio of potassium salt to sodium salt 1:0.092. The active-ingredient content of the solubilized mass (relative to the starting content of ibuprofen of 44.16%) was 103.1%. Due to the more severe reaction conditions which are necessary due to the short residence time in the extruder, the reaction was less unequivocal and some of the formed sodium hydrogen carbonate and any small quantities of formed potassium hydrogen carbonate changed into C0₂ and water.

The scanning electron microscopic examination of the granule showed that the granule had a honeycomb-like structure, with voids of approx. 30 μm diameter. These were obviously trapped CO₂ microbubbles. This is also the reason why this extruded granule is extremely suitable for the preparation of sachets or stickpacks. If this granule was shaken into water, after a few seconds it began to float on the surface of the water while dissolving, clearly because of the trapped carbon dioxide.

The extruded granule with an average grain of 0.8 mm dissolved within 26 seconds under the conditions described in example 2.

### Example 7

### Preparation of an Effervescent Granule

The following were loaded into a vacuum granulator

| | |
|---|---|
| 105.8 kg | citric acid |
| 133.4 kg | monosodium citrate |
| 501.4 kg | sodium hydrogen carbonate |
| 18.4 kg | saccharine sodium |
| 4.6 kg | sucrose palmitate |
| 13.8 kg | hydroxypropyl methylcellulose |

and mixed for 10 minutes. 4.6 kg ethanol was sprayed into the mixture with a single-substance nozzle and the whole mixture stirred for a further 15 minutes. A fine-structured granule formed. The water jacket of the vacuum granulator was set at 60° C. and the vacuum at approx. 100 mbar. After a drying time of approx. 75 minutes the product was cooled to approx. 30-35° C. and the effervescent granule removed. The granule was screened through a sieve with a mesh width of 1.25 mm and stored in tight-closing containers.

### Example 8

### Preparation of Effervescent Tablets

In a suitable mixing vessel 710 kg ibuprofen granules according to example 1, 1690 kg effervescent granules according to example 7, 250 kg sodium hydrogen carbonate, 100 kg citric acid, 10 kg menthol flavouring (163592 Symrise), 40 kg grapefruit flavouring (3018177 Symrise) were poured in and mixed for 15 minutes.

The following effervescent tablets were prepared from the mixture in known technical manner:

| | Ibuprofen | Diameter | Tablet weight |
|---|---|---|---|
| Tablet | content [mg] | [mm] | [mg] |
| 1 | 200 | 18 | 1400 |
| 2 | 400 | 25 | 2800 |
| 3 | 600 | 25 | 4200 |

All tablets dissolved clear within 2-3.5 minutes. The hardness of the tablets was 50 N for the 200-mg effervescent tablet and approx. 60-90 N for the 400- and 600-mg effervescent tablets. After the named decomposition time practically no sediment was visible. The decomposition of the tablets was accompanied by an immediate and complete dissolution of the granules found in the tablets. Practically no coarse particles of active ingredient which would be still visible after the decomposition of the effervescent tablet formed. Particularly noteworthy is the taste of the 200-mg effervescent tablets, outstanding compared with competing tablets in the market, and the clear dissolution without visible, floating particles of active ingredient.

### Example 9

### Preparation of Drinkable Granule

400 kg ibuprofen granule according to example 6 was mixed with 80 kg effervescent granule according to example 7. 10 kg menthol flavouring (163592 Symrise) and 40 kg grapefruit flavouring (301877 Symrise) were added to the mixture. The mixture was packed in so-called stickpacks with the following weights:
200 mg dose: 601 mg
400 mg dose: 1202 mg
600 mg dose: 1803 mg
800 mg dose: 2404 mg

If the contents of a stick were shaken into a glass with 200 ml water, 20° C., all the doses dissolved of their own accord without stirring within 30-90 seconds and were thereafter ready to drink.

1. Effervescent ibuprofen formulation containing
(a) an active ingredient-containing granule which contains ibuprofen and a basic excipient, and
(b) an effervescent granule which contains an acid component and a carbon dioxide-forming component.
characterized in that the active ingredient-containing granule contains the ibuprofen in the form of a water-soluble salt.

2. Effervescent formulation according to claim 1, in which the active ingredient-containing granule contains the potassium salt of ibuprofen.

3. Effervescent formulation according to claim 1, in which the active ingredient-containing granule contains the sodium salt of ibuprofen.

4. Effervescent formulation according to claim 3, which contains the potassium salt and the sodium salt of ibuprofen in a molar ratio of potassium to sodium salt of 1:0 to 1:1.

5. Effervescent formulation according to claim 1, in which the basic component of the active ingredient-containing granules contains NaHCO₃, KHCO₃, Na₂CO₃, K₂CO₃, Na₂HPO₄, K₂HPO₄, disodium citrate, dipotassium citrate, the protonated form of lysine, arginine, potassium glycinate, sodium glycinate, meglumine or a mixture thereof.

6. Effervescent formulation according to claim 5, in which the basic component of the active ingredient-containing granule contains KHCO₃ or a mixture of KHCO₃ and NaHCO₃.

7. Effervescent formulation according to claim 1, in which the ratio of ibuprofen salt to basic excipient in the active ingredient-containing granule lies in the range from 0.8 to 2 mol basic excipient per mol ibuprofen salt.

8. Effervescent formulation according to claim 1, in which the effervescent granule contains as acid component citric acid, tartaric acid, malic acid, ascorbic acid, an acid salt of these acids, an acid inorganic salt, glutamic acid, adipic acid, glutamic acid hydrochloride, betaine hydrochloride or a mixture thereof.

9. Effervescent formulation according to claim 1, in which the effervescent granule contains as carbon dioxide-forming component an alkaline or alkaline-earth hydrogen carbonate or carbonate.

10. Effervescent formulation according to claim 1 in the form of a dosing unit.

11. Effervescent formulation according to claim 10 in the form of an effervescent tablet on granule poured into sachets or stickpacks.

12. Effervescent formulation according to claim 10, which contains 200 to 800 mg ibuprofen (measured as free acid) per dosing unit.

13. Effervescent formulation according to claim 10 in the form of an effervescent tablet, wherein an effervescent tablet has a total weight of 1000 to 1500 mg per 200 mg ibuprofen (measured as free acid).

14. Effervescent formulation according to claim 10 in the form of a drinkable granule, wherein a dosing unit of the drinkable granule has a total weight of 500 to 950 mg per 200 mg ibuprofen (measured as free acid).

15. Effervescent formulation according to claim 1 which contains 2 to 4 parts by weight alkali hydrogen carbonate relative to one part by weight ibuprofen.

16. Effervescent formulation according to claim 1, in which the active ingredient-containing granule (a) can be prepared according to a process of the following claims.

17. Process for the preparation of an effervescent formulation according to claim 1, in which the active ingredient-containing granule is prepared by intensively mixing ibuprofen in the form of its free acid with one or more basic excipients without adding water.

18. Process according to claim 17, in which the ibuprofen and the basic excipient are mixed together accompanied by the addition of an organic solvent.

19. Process according to claim 18, in which isopropanol, ethanol, acetone or a mixture thereof is added as organic solvent.

20. Process according to claim 18, in which the organic solvent is added in a quantity of 0.5 to 10 wt.-% relative to the total weight of the reaction mixture.

21. Process according to claim 17, in which the mixture of ibuprofen and excipients is heated to a temperature of 50 to 60° C.

22. Process according to claim 17, in which a water-soluble basic substance which has a pH>11.0 in 0.1 molar aqueous solution is used as basic excipient.

23. Process according to claim 22, in which a basic substance is used which is selected from tripotassium phosphate, trisodium phosphate, tripotassium citrate, trisodium citrate and the corresponding sodium salts, the potassium and sodium salt of glycine, lysine, arginine, physiologically acceptable organic amines, meglumine, alkali carbonates and mixtures of these substances.

24. Process according to claim 23, in which potassium carbonate, sodium carbonate or a mixture thereof is used as basic excipient.

25. Process according to claim 17, in which a mixture of potassium and sodium compounds is used in a molar ratio (K:Na) of 1:0 to 1:1 as basic excipient.

26. Process according to claim 17, in which 0.8 to 2 mol basic excipient is used per mol ibuprofen.

27. Process according to claim 17 in which the mixing is carried out in a vacuum granulator, compactor, fluid-bed reactor or extruder.

28. Process according to claim 1, in which after its preparation the granule is dried to a water content of less than 0.5 wt.-%.

29. Process according to claim 28, in which the granule is dried to a water content of less than 0.25 wt.-%.

30. Process according to claim 17, in which the active ingredient-containing granule is then mixed with an effervescent granule.

31. Process according to claim 30, in which the mixture of active ingredient-containing granule and effervescent granule is packed in sachets or stickpacks or compressed into tablets.
